(19) United States
(12) Patent Application Publication
(10) Pub. No.: US 2022/0341801 A1
(43) Pub. Date: Oct. 27, 2022

(54) ELECTRONIC VAPORIZING DEVICE CHIP WITH AIR PRESSURE SENSING UNIT AND WORKING METHOD THEREOF

(71) Applicant: SHENZHEN HAPPY VAPING TECHNOLOGY LIMITED, Shenzhen, Guangdong (CN)

(72) Inventors: Guangrong Lin, Shenzhen, Guangdong (CN); Xiyong Zhang, Shenzhen, Guangdong (CN); Xianbin Zheng, Shenzhen, Guangdong (CN)

(21) Appl. No.: 17/762,729

(22) PCT Filed: Sep. 17, 2020
(86) PCT No.: PCT/CN2020/115735

§ 371 Date: Mar. 23, 2022

(30) Foreign Application Priority Data
Nov. 5, 2019 (CN) 201911071157.2

(51) Int. Cl. G01L 9/06; H05B 1/02; G01L 19/00; A24F 40/51; A24F 40/65

(52) U.S. Cl. CPC G01L 9/06; H05B 1/0244; G01L 19/0092; A24F 40/51; A24F 40/65

(57) ABSTRACT

Disclosed is an electronic vaporizing device chip with air pressure sensing unit and working method thereof, where the chip includes an air pressure sensing unit, a control unit, a plurality of auxiliary resistors, a capacitor, and a plurality of pins, the air pressure sensing unit is configured to detect an air pressure generated inside the electronic vaporizing device during suction, a signal amplification module is configured to amplify and then transmit an air pressure analog signal detected by the air pressure sensing unit to an analog-to-digital conversion module, the analog-to-digital conversion module converts the air pressure analog signal into an air pressure digital signal, a data processing and calibration module processes and calibrates the air pressure digital signal and then converts it into an air flow quantity digital signal; the advantage is that an air pressure sensing function and an air flow quantity calculating function are combined.

### TECHNICAL FIELD

The present disclosure relates to a technical field of the electronic vaporizing device, and more particularly, the present disclosure relates to an electronic vaporizing device chip with an air pressure sensing unit and a working method of the electronic vaporizing device chip with an air pressure sensing unit.

### BACKGROUND

The existing vaporized electronic cigarettes are usually heated by the vaporizer to vaporize the electronic cigarette liquid to generate vapor for the smoker to use. Since the electronic cigarette liquid does not contain the cigarette tar, the harm to the human body is avoided. Thus, the electronic cigarettes are widely used and gradually replace tobacco cigarettes.

The Chinese patent with application No. 201910269123.8, publication No. 109924548A, entitled “vaporizing device capable of controlling administration amount, and control method for same” discloses a vaporizing device capable of controlling administration amount. The vaporizing device comprises a battery, a control circuit board, a heating resistor, a vaporizing passage, a mouth piece, a detecting air passage, and an air pressure sensor. The control circuit board is arranged with a microcontroller, an air flow quantity calculating unit, an energy statistics unit, an energy and vaporizing amount conversion unit, and a power control unit, which are electrically connected with each other. The air flow quantity calculating unit calculates corresponding air flow quantity based on the modeling volume of the detecting air passage and a change of suction pressure. The power control unit controls a power output to the heating resistor based on the air flow quantity. The energy statistics unit calculates output energy based on the output power and time. The energy and vaporizing amount conversion unit calculates corresponding value of aerosol intake from the energy. The patent discloses that the change of the suction pressure is detected by the air pressure sensor, and then the change of the suction pressure is converted into the air flow quantity by the air flow quantity calculation unit, and finally the purpose of limiting the intake of aerosol is achieved.

In the above patent, the air pressure sensor and the air flow quantity calculation unit belong to separate components and circuit units respectively, the circuit structure is complex, the volume thereof is large, the signal standard is not uniform, and the conversion takes a long time, so data transmission between the two has disadvantages of a slow response and a large error, and at the same time, a control accuracy is reduced.

### SUMMARY

### Technical Problem

An object of the present disclosure is to provide an electronic vaporizing device chip with air pressure sensing unit and working method thereof in order to overcome the deficiencies of the above-mentioned technology. The electronic vaporizing device chip with an air pressure sensing unit combines the air pressure sensing function and the air flow quantity calculating function into one.

### Technical Solution

The technical solution of the present disclosure is achieved as follows, an electronic vaporizing device chip with an air pressure sensing unit, comprising an air pressure sensing unit, a control unit, a plurality of auxiliary resistors, a capacitor, and a plurality of pins, wherein the air pressure sensing unit, the control unit, the plurality of auxiliary resistors, the capacitor, and the plurality of pins are electrically connected. The control unit comprises a memory, a control logic module, an air pressure sensing unit driving module, a signal amplification module, an analog-to-digital conversion module, a data processing and calibration module, a communication interface module, and an enabling signal input control circuit. The air pressure sensing unit is configured to detect an air pressure generated inside the electronic vaporizing device when suction, the memory is configured to store a relevant parameter, the control logic module is configured for a logic control of an internal circuit of the control unit, the air pressure sensing unit driving module is configured to drive the air pressure sensing unit to work, the signal amplification module is configured to amplify and then transmit an air pressure analog signal detected by the air pressure sensing unit to the analog-to-digital conversion module, the analog-to-digital conversion module converts the air pressure analog signal into an air pressure digital signal, the data processing and calibration module processes and calibrates the air pressure digital signal and then converts it into an air flow quantity digital signal, the communication interface module is configured for a communication connection with an external component, and the enabling signal input control circuit is configured to receive an external enabling signal and control the communication interface module to work.

Preferably, the control unit is inactive in a standby state, the air pressure sensing unit is configured to have a patrol detection for the air pressure, and the control unit immediately enters a working state once a change of the air pressure is detected.

Preferably, the plurality of pins comprise at least a power supply pin, a ground pin, an activation signal output pin, an internal data output pin, an external data input pin, a clock signal pin, and an enabling signal input pin. The activation signal output pin serves to output an activation signal generated by the control unit to an external controller so as to activate the electronic vaporizing device. The internal data output pin, the external data input pin, and the clock signal pin are connected to the communication interface module, the internal data output pin serves to output an internal data to the external controller, and the external data input pin serves to input external data, the clock signal pin serves to input a clock signal to read and write data, and the enabling signal input pin serves to input an enabling signal to control an output of the internal data and an input of the external data.

Preferably, an air pressure change threshold value is preset in the memory, and the control unit is configured to generate the activation signal in a condition that the control unit determines that a change value of the air pressure digital signal reaches the preset air pressure change threshold value.

Preferably, the electronic vaporizing device chip with an air pressure sensing unit, further comprising a temperature sensing unit for detecting temperature of the electronic vaporizing device chip, wherein the control unit further comprises a circuit switching module for switching the air pressure analog signal detected by the air pressure sensing unit and a temperature analog signal detected by the temperature sensing unit, and then transmitting the switched air pressure analog signal or the switched temperature analog signal to the analog-to-digital conversion module in turn, and the analog-to-digital conversion module may be capable of convert the temperature analog signal into a temperature digital signal.

Preferably, the control unit further comprises an output control module electrically connected with the data processing and calibration module, the output control module is configured to generate a PWM output control signal based on the air flow quantity digital signal for controlling power of the electronic vaporizing device.

Preferably, the pins further comprise a PWM output control signal pin, and the PWM output control signal pin serves to output the PWM output control signal generated by the output control module.

Preferably, the control unit further comprises an on-chip oscillator for providing a frequency signal for the control unit during working.

Preferably, the control unit further comprises a real-time clock module for timing.

Another technical solution of the present disclosure is a working method of an electronic vaporizing device chip with an air pressure sensing unit, wherein the working method comprises steps of:

(1) initializing a relevant parameter when the chip is powered;

(2) activating an air pressure sensing unit when the chip is in a standby state;

(3) determining whether the air pressure sensing unit detects a change of an air pressure analog signal of the electronic vaporizing device, if yes, go to next step, if no, return to previous step;

(4) amplifying the air pressure analog signal by a signal amplification module and converting the amplified air pressure analog signal into an air pressure digital signal by an analog-to-digital conversion module;

(5) processing and calibrating the air pressure digital signal by a data processing and calibration module and converting the processed and calibrated air pressure digital signal into a corresponding air flow quantity digital signal;

(6) by means of a control logic module, determining whether a change value of the air pressure digital signal reaches a set air pressure change threshold value, if yes, simultaneously go to steps (7) and (8), if no, return to step (2);

(7) generating an activation signal by a control unit and outputting the activation signal to an external controller through an activation signal output terminal to activate the electronic vaporizing device;

(8) determining whether an enabling signal input control circuit receives an enabling signal and whether a communication interface module receives a data transmission request signal, if both yes, go to next step, if no, return to step (2); and

(9) outputting the air flow quantity digital signal to the external controller through an internal data output pin by the communication interface module, and then returning to step (2).

Another technical solution of the present disclosure is a working method of an electronic vaporizing device chip with an air pressure sensing unit, wherein the working method comprises steps of:

(1) initializing a relevant parameter when the chip is powered;

(2) activating an air pressure sensing unit and a temperature sensing unit when the chip is in a standby state, to simultaneously go to steps (3) and (4);

(3) determining whether the air pressure sensing unit detects a change of an air pressure analog signal of the electronic vaporizing device, if yes, go to step (5), if no, return to step (2);

(4) detecting a temperature analog signal of an internal temperature of the chip by the temperature sensing unit;

(5) switching the air pressure analog signal and the temperature analog signal in turn by a circuit switching module, if being switched to the air pressure analog signal, go to step (6); if being switched to the temperature analog signal, go to step (12);

(6) amplifying the air pressure analog signal by a signal amplification module and converting the amplified air pressure analog signal into an air pressure digital signal by an analog-to-digital conversion module;

(7) processing and calibrating the air pressure digital signal by a data processing and calibration module and converting the processed and calibrated air pressure digital signal into a corresponding air flow quantity digital signal;

(8) by means of a control logic module, determining whether a change value of the air pressure digital signal reaches a set air pressure change threshold value, if yes, simultaneously go to steps (9), (10), and (12), if no, return to step (2);

(9) generating an activation signal by a control unit and outputting the activation signal to an external controller through an activation signal output terminal to activate the electronic vaporizing device;

(10) determining whether the air flow quantity digital signal reaches a set minimum air flow quantity threshold value, if yes, go to next step, if no, return to step (2);

(11) generating and outputting a PWM output control signal by an output control module, then returning to step (2);

(12) determining whether an enabling signal input control circuit receives an enabling signal and whether a communication interface module receives a data transmission request signal, if both yes, go to next step, if no, return to step (2);

(13) outputting the air flow quantity digital signal to the external controller through an internal data output pin by the communication interface module, then returning to step (2);

(14) amplifying the temperature analog signal by the signal amplification module and converting the amplified temperature analog signal into a temperature digital signal by the analog-to-digital conversion module;

(15) by means of the control logic module, determining whether the temperature digital signal reaches a set temperature threshold value, if yes, go to next step, if no, return to step (2); and

(16) inactivating the chip and providing high temperature or low temperature protection.

### Beneficial Effect

The electronic vaporizing device chip with an air pressure sensing unit has a built-in air pressure sensing unit and a control unit. The control unit directly amplifies, converts, and calibrates an air pressure signal detected by the air pressure sensing unit inside the chip through a control logic module, a signal amplification module, an analog-to-digital conversion module, and a data processing and calibration module, and outputs an air flow quantity signal. In this way, a built-in air pressure signal acquisition circuit and a high-accuracy analog-to-digital converter allow an accurate acquisition of the air pressure signal, the built-in control unit allows an accurate calibration and compensation of the signal, and a built-in algorithm allows an accurate conversion of the pressure to the air flow. Therefore, a data transmission response can be fast, and the air pressure signal can be accurately converted inside the chip, which will greatly improve a control accuracy of a vaporizing amount of the electronic vaporizing device. At the same time, a customizable air pressure change threshold value is provided, and an activation signal is given when a change of the air pressure is greater than a set threshold value, in such a case, a user's vaping action may be accurately judged and a probability of a false activation of the electronic vaporizing device may be reduced. In addition, the chip has a high integration level, so that there are few external components, and the chip has high work efficiency, with less amount of heat being generated. Due to the small size, the chip can be very easily installed on a circuit board of the electronic vaporizing device which has very small space.

### BRIEF DESCRIPTION OF DRAWINGS

FIG. 1 is a first internal circuit structure diagram of an electronic vaporizing device chip according to an embodiment of the present disclosure;

FIG. 2 is a second internal circuit structure diagram of an electronic vaporizing device chip according to an embodiment of the present disclosure;

FIG. 3 is a first flowchart of a working method of an electronic vaporizing device chip according to an embodiment of the present disclosure;

FIG. 4 is a second flowchart of a working method of an electronic vaporizing device chip according to an embodiment of the present disclosure.

### DETAILED DESCRIPTION

In order to make the objects, technical solutions, and advantages of the present disclosure clearer, the present disclosure will be further described in detail below with reference to the accompanying drawings and embodiments. It should be understood that the specific embodiments described herein are merely used for explaining the present disclosure, and are not intended to limit the present disclosure.

The electronic vaporizing device mentioned in the present disclosure is a vaporizing device or a vaporizer that heats and evaporates a liquid substance into a vaporous mist-like substance. The electronic vaporizing device can not only be used for electronic cigarettes to vaporize electronic cigarette liquid to generate vapor, but also can be used in the medical field, that is, a liquid in which drugs are dissolved can be heated and evaporated into a mist-like substance for patients to vape, so as to achieve the purpose of treatment.

### Embodiment 1

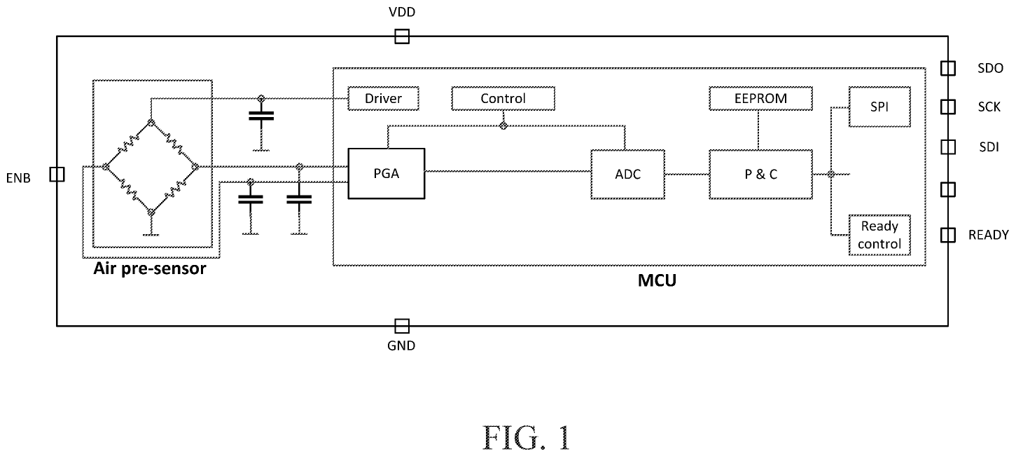

As shown in FIG. 1, an electronic vaporizing device chip with an air pressure sensing unit of the present disclosure, an internal package of which comprising an air pressure sensing unit Airpre-sensor, a control unit MCU, a plurality of auxiliary resistors ( not shown in the figure), a capacitor C, and a plurality of pins provided on sides of the chip, wherein the air pressure sensing unit Airpre-sensor, the control unit MCU, the plurality of auxiliary resistors (not shown in the figure), the capacitor C, and the plurality of pins provided on sides of the chip are electrically connected. The control unit MCU comprises a memory EEPROM, a control logic module Control, an air pressure sensing unit driving module Driver, a signal amplification module PGA, an analog-to-digital conversion module ADC, a data processing and calibration module P&C, a communication interface module SPI, and an enabling signal input control circuit Ready control. The air pressure sensing unit Airpre-sensor is configured to detect an air pressure generated inside the electronic vaporizing device when suction. The suction generally generates a negative pressure, and the air pressure sensing unit Airpre-sensor obtains an air pressure analog signal after detecting.

The memory EEPROM is configured to store a set relevant parameter and a relevant parameter generated during working, the control logic module Control is configured for a logic control, an analysis, and a judgment of an internal circuit of the control unit MCU, the air pressure sensing unit driving module Driver is configured to assist and drive the air pressure sensing unit Airpre-sensor to work, the signal amplification module PGA is configured to amplify the air pressure analog signal detected by the air pressure sensing unit Airpre-sensor and transmit it to the analog-to-digital conversion module ADC, the analog-to-digital conversion module ADC converts the air pressure analog signal into an air pressure digital signal, and the data processing and calibration module P&C processes and calibrates the air pressure digital signal and then converts it into an air flow quantity digital signal. Since the external air pressure is not stable and constant, calibration is needed to avoid errors.

The communication interface module SPI and the enabling signal input control circuit Ready control connect some of the plurality of the pins. The communication interface module SPI is used for a communication connection with an external component and input or output a data signal, and the enabling signal input control circuit Ready control is configured to receive an external enabling signal and control the communication interface module SPI to work, that is, when the enabling signal is received, the enabling signal input control circuit Ready control enable the communication interface module SPI to output data or input data.

In order to reduce an energy consumption of the electronic vaporizing device during standby, the chip of the present disclosure is also provided with a standby state. The control unit MCU is inactive in the standby state, and the air pressure sensing unit Airpre-sensor is configured to have a patrol detection for the air pressure, so as to save power. When the air pressure sensing unit Airpre-sensor detects that the air pressure changes, the control unit MCU immediately enters a working state. When a suction occurs, a negative pressure will be generated inside the electronic vaporizing device, that is, the pressure will change. When the air pressure sensing unit Airpre-sensor detects a change of the air pressure analog signal inside the electronic vaporizing device, the control unit MCU is woken up, powered and enters the working state.

As shown in FIG. 1, the pins of the chip comprise a power supply pin VDD, a ground pin GND, an activation signal output pin ENB, an internal data output pin SDO, an external data input pin SDI, a clock signal pin SCK, and an enabling signal input pin READY. The activation signal output pin ENB serves to output an activation signal generated by the control unit MCU to an external controller (not shown in the figure) in order to activate the electronic vaporizing device. The internal data output pin SDO, the external data input pin SDI, and the clock pin SCK are connected to the communication interface module SPI. The internal data output pin SDO serves to output internal data to the external controller, the external data input pin SDI serves to input external data, and the clock signal pin SCK serves to input a clock signal to read and write data. The enabling signal input pin READY serves to input the enabling signal to control the internal data output and the external data input.

As shown in FIG. 1, an air pressure change threshold value is preset in the memory EEPROM, and the control unit MCU is configured to generate the activation signal in a condition that the control unit MCU determines that a change value of the air pressure digital signal reaches the set air pressure change threshold value. That is, when the control unit MCU determines that the change value of the air pressure digital signal reaches the set air pressure change threshold value, the control unit MCU generates the activation signal to be output to the external controller, thereby activating the electronic vaporizing device. Under normal circumstances, a change of an internal pressure of the electronic vaporizing device unintentionally caused by a user, and a change of the external air pressure may falsely trigger the electronic vaporizing device to enter the working state. In order to avoid this situation, it is necessary to preset the air pressure change threshold value. When a value of the change of the internal pressure of the electronic vaporizing device exceeds the preset air pressure change threshold value, it is determined that the electronic vaporizing device needs to be started to work. Otherwise, even if there is a slight change of the air pressure, the electronic vaporizing device will not be activated by false triggering.

The chip of the embodiment is characterized in that it is packaged with an air pressure sensing unit Airpre-sensor, a control unit MCU, and a plurality of peripheral auxiliary resistors, and an auxiliary capacitor. The air pressure sensing unit Airpre-sensor and the control unit MCU can be set as two built-in bare chips. The air pressure sensing unit Airpre-sensor is a sensor that uses a pressure sensitive element, that is, a bridge pressure sensor to detect a change value of an air pressure, and the change value of the air pressure could be converted to corresponding air flow quantity. A piezoresistive pressure sensor is integrated into a single chip of the embodiment, and a sensor signal conditioning and transmission circuit with the control unit MCU as the core is integrated into the chip as well, and an SPI interface is used for output. The chip of the embodiment detects the air flow quantity by detecting a change of the air pressure, a built-in signal acquisition circuit and a high-accuracy analog-to-digital converter allow an accurate acquisition of a signal of a sensing unit, and the built-in MCU allows a calibration and compensation of the signal, a built-in algorithm allows a conversion of pressure to airflow, and measurement data is provided synchronously through the SPI interface and sent to an external component. The chip has high flexibility, and algorithms and functions thereof can be extended.

### Embodiment 2

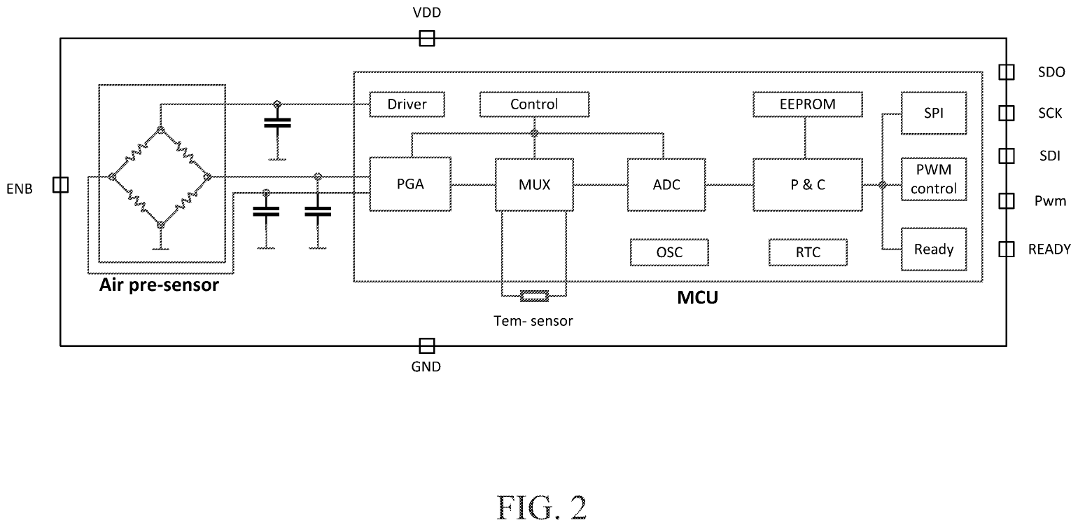

As shown in FIG. 2, on the basis of the foregoing embodiment 1, the electronic vaporizing device chip of this embodiment comprises, in addition to the circuit structural components described in embodiment 1, a temperature sensing unit Tem-sensor for detecting a temperature of the electronic vaporizing device chip. The control unit MCU also comprises a circuit switching module MUX, which is configured to switch the air pressure analog signal detected by the air pressure sensing unit Airpre-sensor and a temperature analog signal detected by the temperature sensing unit Tem-sensor in turn, and then transmit the switched air pressure analog signal or the switched temperature analog signal to the analog-to-digital conversion module ADC in turn. The analog-to-digital conversion module ADC can also convert the temperature analog signal into a temperature digital signal. The purpose of providing the temperature sensing unit Tem-sensor is to detect a temperature of the chip during working, prevent abnormal temperature caused by the chip working under high load or in a high temperature or low temperature environment, and avoid chip damage and failure.

As shown in FIG. 2, the control unit MCU further comprises an output control module PWM control electrically connected with the data processing and calibration module P&C, and the output control module PWM control is configured to generate a PWM output control signal. The pins also comprise a PWM output control signal pin PWM, and the PWM output control signal pin serves to output a PWM output control signal generated by the output control module PWM control. The output control module PWM control is configured to generate the PWM output control signal based on the air flow quantity digital signal for controlling power of the electronic vaporizing device.

As shown in FIG. 2, the control unit MCU further comprises an on-chip oscillator OSC for providing a frequency signal when the control unit MCU works. The control unit MCU also comprises a real-time clock module RTC for timing and reading time.

The chip of the embodiment is characterized in that it is packaged with an air pressure sensing unit Airpre-sensor, a control unit MCU, and a plurality of peripheral auxiliary resistors, and an auxiliary capacitor. The air pressure sensing unit Airpre-sensor and the control unit MCU can be set as two built-in bare chips. The air pressure sensing unit Airpre-sensor is a sensor that uses a pressure sensitive element, that is, a bridge pressure sensor to detect a change value of an air pressure, and the change value of the air pressure could be converted to corresponding air flow quantity. A piezoresistive pressure sensor and a temperature sensor are integrated into a single chip of the embodiment, an air pressure sensing unit signal conditioning and transmission circuit with the control unit MCU as the core is integrated into the chip as well, an SPI interface is used for output, and a PWM signal output is also supported. The chip of the embodiment detects the air flow quantity by detecting a change of the air pressure, a built-in signal acquisition circuit and a high-accuracy analog-to-digital converter allow an accurate acquisition of a signal of a sensing unit, and the built-in MCU allows a calibration and compensation of the signal, a built-in algorithm allows a conversion of pressure to airflow. At the same time, a customizable air flow quantity threshold value is provided, a PWM control signal is given when the air flow quantity is greater than a set minimum air flow quantity threshold value, and measurement data is provided synchronously through the SPI interface and sent to an external component. The chip has high flexibility, and algorithms and functions can be extended.

Embodiment 1 of working methods of the chip:

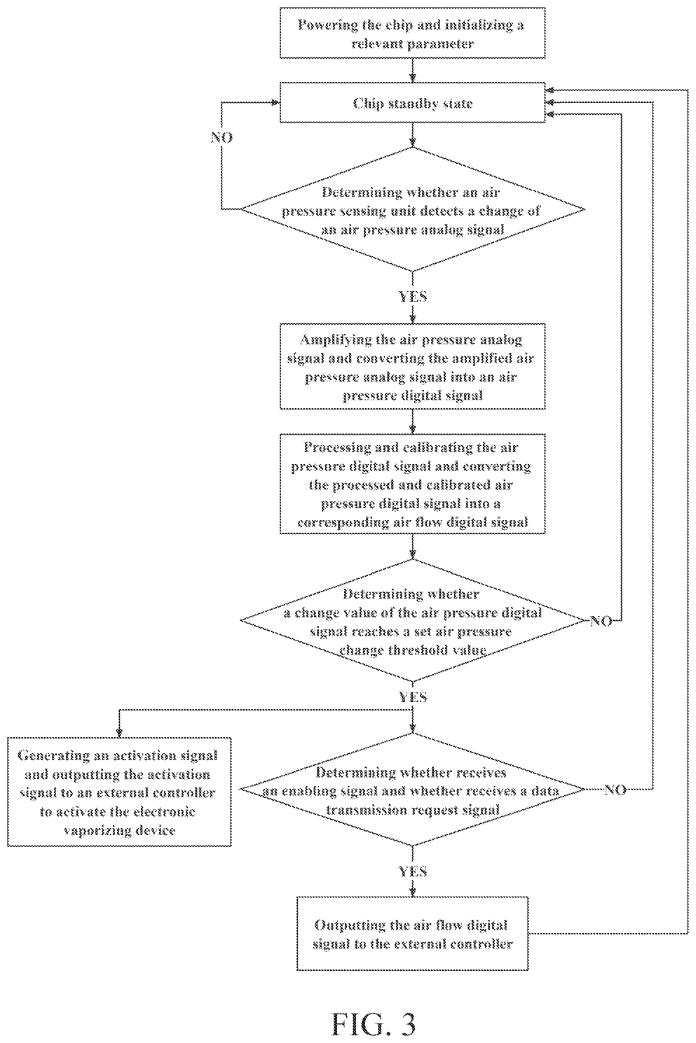

As shown in FIG. 3, a working method of an electronic vaporizing device chip with an air pressure sensing unit, wherein the working method comprises steps of:

(1) initializing a relevant parameter when the chip is powered;

(2) activating an air pressure sensing unit when the chip is in a standby state and a control unit is inactive;

(3) determining whether the air pressure sensing unit detects a change of an air pressure analog signal of the electronic vaporizing device, that is, detecting whether the electronic vaporizing device has an action of suction, if yes, go to next step, if no, return to previous step;

(4) activating the control unit, and amplifying the air pressure analog signal by a signal amplification module and converting the amplified air pressure analog signal into an air pressure digital signal by an analog-to-digital conversion module;

(5) processing and calibrating the air pressure digital signal by a data processing and calibration module into an accurate air pressure digital signal, and converting the accurate air pressure digital signal into a corresponding air flow quantity digital signal;

(6) by means of a control logic module, determining whether a change value of the air pressure digital signal reaches a set air pressure change threshold value, that is, determining whether the action of suction is caused by a false trigger action, if yes, that is, the action of suction is not caused by the false trigger action, simultaneously go to steps (7) and (8), if no, that is, the action of suction may be caused by the false trigger action, return to step (2);

(7) generating an activation signal by a control unit and outputting the activation signal to an external controller through an activation signal output terminal to activate the electronic vaporizing device;

(8) determining whether an enabling signal input control circuit receives an enabling signal and whether a communication interface module receives a data transmission request signal, if both yes, go to next step, if no, return to step (2); and

(9) outputting the air flow quantity digital signal to the external controller through an internal data output pin by the communication interface module, and then returning to step (2).

For the names of the components or modules involved in the above steps, referring to Embodiment 1 and FIG. 1, in the above steps, the air pressure sensing unit continuously detects whether the air pressure changes.

Embodiment 2 of the working methods of the chip:

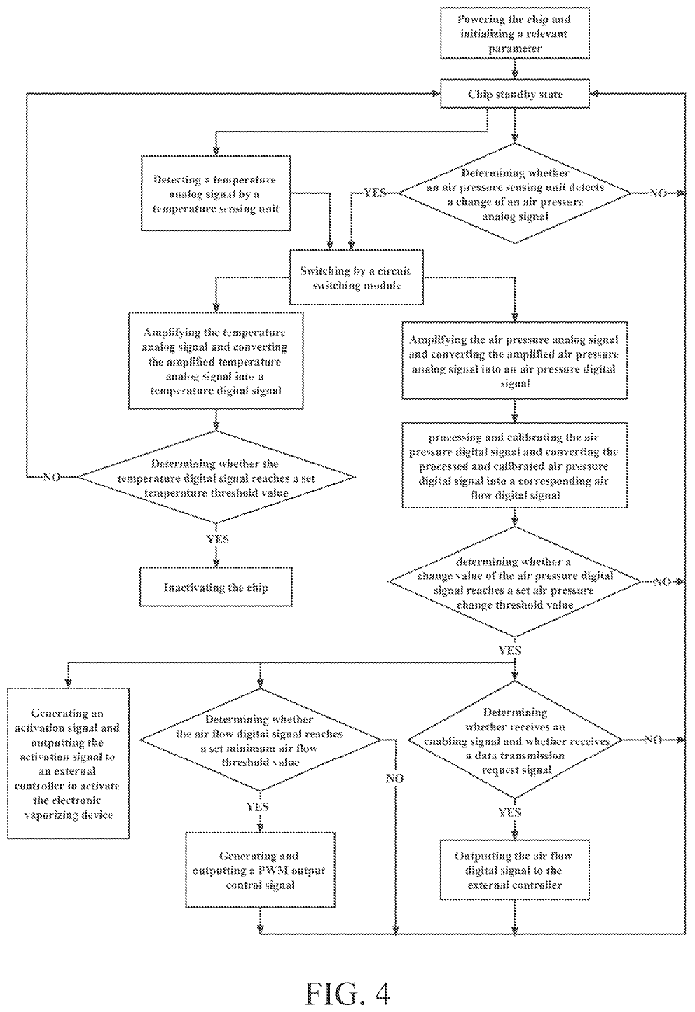

As shown in FIG. 4, a working method of an electronic vaporizing device chip with an air pressure sensing unit, wherein the working method comprises steps of:

(1) initializing a relevant parameter when the chip is powered;

(2) activating an air pressure sensing unit and a temperature sensing unit when the chip is in a standby state, to simultaneously go to steps (3) and (4);

(3) determining whether the air pressure sensing unit detects a change of an air pressure analog signal of the electronic vaporizing device, that is, detecting whether the electronic vaporizing device has an action of suction, if yes, go to step (5), if no, return to step (2);

(4) detecting a temperature analog signal of an internal temperature of the chip by the temperature sensing unit;

(5) switching the air pressure analog signal and the temperature analog signal in turn by a circuit switching module, if being switched to the air pressure analog signal, go to step (6); if being switched to the temperature analog signal, go to step (12);

(6) amplifying the air pressure analog signal by a signal amplification module and converting the amplified air pressure analog signal into an air pressure digital signal by an analog-to-digital conversion module;

(7) processing and calibrating the air pressure digital signal by a data processing and calibration module into an accurate air pressure digital signal, and converting the accurate air pressure digital signal into a corresponding air flow quantity digital signal;

(8) by means of a control logic module, determining whether a change value of the air pressure digital signal reaches a set air pressure change threshold value, that is, determining whether the action of suction is caused by a false trigger action, if yes, that is, the action of suction is not caused by the false trigger action, simultaneously go to steps (9), (10), and (12), if no, that is, the action of suction may be caused by the false trigger action, return to step (2);

(9) generating an activation signal by a control unit and outputting the activation signal to an external controller through an activation signal output terminal to activate the electronic vaporizing device;

(10) determining whether the air flow quantity digital signal reaches a set minimum air flow quantity threshold value, that is, determining whether the action of suction is caused by a false trigger action, if yes, that is, the action of suction is not caused by the false trigger action, go to next step, if no, that is, the action of suction may be caused by the false trigger action, return to step (2);

(11) generating and outputting a PWM output control signal by an output control module, then returning to step (2);

(12) determining whether an enabling signal input control circuit receives an enabling signal and whether a communication interface module receives a data transmission request signal, if both yes, go to next step, if no, return to step (2);

(13) outputting the air flow quantity digital signal to the external controller through an internal data output pin by the communication interface module, then returning to step (2);

(14) amplifying the temperature analog signal by the signal amplification module and converting the amplified temperature analog signal into a temperature digital signal by the analog-to-digital conversion module;

(15) by means of the control logic module, determining whether the temperature digital signal reaches a set temperature threshold value, if yes, go to next step, if no, return to step (2); and

(16) inactivating the chip and providing high temperature or low temperature protection, the air pressure sensing unit and the control unit are both inactive at this time, and after a time of being protected from high temperature or low temperature is over, reactivating the chip.

For the names of the components or modules involved in the above steps, referring to Embodiment 2 and FIG. 2, in the above steps, the air pressure sensing unit continuously detects whether the air pressure changes.

### INDUSTRIAL APPLICABILITY

The above descriptions are only preferred embodiments of the present disclosure, and all equivalent changes and modifications made according to the scope of the claims of the present disclosure shall fall within the scope of the claims of the present disclosure.

1. An electronic vaporizing device chip with an air pressure sensing unit, comprising an air pressure sensing unit, a control unit, a plurality of auxiliary resistors, a capacitor, and a plurality of pins, wherein the air pressure sensing unit, the control unit, the plurality of auxiliary resistors, the capacitor, and the plurality of pins are electrically connected, the control unit comprises a memory, a control logic module, an air pressure sensing unit driving module, a signal amplification module, an analog-to-digital conversion module, a data processing and calibration module, a communication interface module, and an enabling signal input control circuit, the air pressure sensing unit is configured to detect an air pressure generated inside the electronic vaporizing device during suction, the memory is configured to store a relevant parameter, the control logic module is configured for a logic control of an internal circuit of the control unit, the air pressure sensing unit driving module is configured to drive the air pressure sensing unit to work, the signal amplification module is configured to amplify and then transmit an air pressure analog signal detected by the air pressure sensing unit to the analog-to-digital conversion module, the analog-to-digital conversion module is configured to convert the air pressure analog signal into an air pressure digital signal, the data processing and calibration module is configured to process and calibrate the air pressure digital signal, and then convert the air pressure digital signal into an air flow quantity digital signal, the communication interface module is configured for a communication connection with an external component, and the enabling signal input control circuit is configured to receive an external enabling signal and control the communication interface module to work.

2. The electronic vaporizing device chip with an air pressure sensing unit according to claim 1, wherein the control unit is inactive in a standby state, the air pressure sensing unit is configured to have a patrol detection for the air pressure, and the control unit immediately enters a working state once a change of the air pressure is detected.

3. The electronic vaporizing device chip with an air pressure sensing unit according to claim 1, wherein the plurality of pins comprise at least a power supply pin, a ground pin, an activation signal output pin, an internal data output pin, an external data input pin, a clock signal pin, and an enabling signal input pin, the activation signal output pin serves to output an activation signal generated by the control unit to an external controller so as to activate the electronic vaporizing device, the internal data output pin, the external data input pin, and the clock signal pin are connected to the communication interface module, the internal data output pin serves to output internal data to the external controller, and the external data input pin serves to input external data, the clock signal pin serves to input a clock signal to read and write data, and the enabling signal input pin serves to input an enabling signal to control an output of the internal data and an input of the external data.

4. The electronic vaporizing device chip with an air pressure sensing unit according to claim 3, wherein an air pressure change threshold value is preset in the memory, and the control unit is configured to generate the activation signal in a condition that the control unit determines that a change value of the air pressure digital signal reaches the preset air pressure change threshold value.

5. The electronic vaporizing device chip with an air pressure sensing unit according to claim 1, further comprising a temperature sensing unit for detecting temperature of the electronic vaporizing device chip, wherein the control unit further comprises a circuit switching module for switching the air pressure analog signal detected by the air pressure sensing unit and a temperature analog signal detected by the temperature sensing unit, and then transmitting the switched air pressure analog signal or the switched temperature analog signal to the analog-to-digital conversion module in turn, and the analog-to-digital conversion module is capable of converting the temperature analog signal into a temperature digital signal.

6. The electronic vaporizing device chip with an air pressure sensing unit according to claim 1, wherein the control unit further comprises an output control module electrically connected with the data processing and calibration module, and the output control module is configured to generate a PWM output control signal based on the air flow quantity digital signal for controlling power of the electronic vaporizing device.

7. The electronic vaporizing device chip with an air pressure sensing unit according to claim 6, wherein the pins comprise a PWM output control signal pin serves to output the PWM output control signal generated by the output control module.

8. The electronic vaporizing device chip with an air pressure sensing unit according to claim 1, wherein the control unit further comprises an on-chip oscillator for providing a frequency signal for the control unit during working.

9. The electronic vaporizing device chip with an air pressure sensing unit according to claim 1, wherein the control unit further comprises a real-time clock module for timing.

10. A working method of an electronic vaporizing device chip with an air pressure sensing unit, wherein the method comprises steps of:
(1) initializing a relevant parameter when the chip is powered;
(2) activating an air pressure sensing unit when the chip is in a standby state;
(3) determining whether the air pressure sensing unit detects a change of an air pressure analog signal of the electronic vaporizing device, if yes, go to next step, if no, return to previous step;
(4) amplifying the air pressure analog signal by a signal amplification module and converting the amplified air pressure analog signal into an air pressure digital signal by an analog-to-digital conversion module;
(5) processing and calibrating the air pressure digital signal by a data processing and calibration module and converting the processed and calibrated air pressure digital signal into a corresponding air flow quantity digital signal;
(6) by means of a control logic module, determining whether a change value of the air pressure digital signal reaches a set air pressure change threshold value, if yes, simultaneously go to steps (7) and (8), if no, return to step (2);
(7) generating an activation signal by a control unit and outputting the activation signal to an external controller through an activation signal output terminal to activate the electronic vaporizing device;
(8) determining whether an enabling signal input control circuit receives an enabling signal and whether a communication interface module receives a data transmission request signal, if both yes, go to next step, if no, return to step (2); and
(9) outputting the air flow quantity digital signal to the external controller through an internal data output pin by the communication interface module, and then returning to step (2).

11. A working method of an electronic vaporizing device chip with an air pressure sensing unit, wherein the method comprises steps of:
(1) initializing a relevant parameter when the chip is powered;
(2) activating an air pressure sensing unit and a temperature sensing unit when the chip is in a standby state, to simultaneously go to steps (3) and (4);
(3) determining whether the air pressure sensing unit detects a change of an air pressure analog signal of the electronic vaporizing device, if yes, go to step (5), if no, return to step (2);
(4) detecting a temperature analog signal of an internal temperature of the chip by the temperature sensing unit;
(5) switching the air pressure analog signal and the temperature analog signal in turn by a circuit switching module, if being switched to the air pressure analog signal, go to step (6); if being switched to the temperature analog signal, go to step (12);
(6) amplifying the air pressure analog signal by a signal amplification module and converting the amplified air pressure analog signal into an air pressure digital signal by an analog-to-digital conversion module;
(7) processing and calibrating the air pressure digital signal by a data processing and calibration module and converting the processed and calibrated air pressure digital signal into a corresponding air flow quantity digital signal;
(8) by means of a control logic module, determining whether a change value of the air pressure digital signal reaches a set air pressure change threshold value, if yes, simultaneously go to steps (9), (10), and (12), if no, return to step (2);
(9) generating an activation signal by a control unit and outputting the activation signal to an external controller through an activation signal output terminal to activate the electronic vaporizing device;
(10) determining whether the air flow quantity digital signal reaches a set minimum air flow quantity threshold value, if yes, go to next step, if no, return to step (2);
(11) generating and outputting a PWM output control signal by an output control module, then returning to step (2);
(12) determining whether an enabling signal input control circuit receives an enabling signal and whether a communication interface module receives a data transmission request signal, if both yes, go to next step, if no, return to step (2);
(13) outputting the air flow quantity digital signal to the external controller through an internal data output pin by the communication interface module, then returning to step (2);
(14) amplifying the temperature analog signal by the signal amplification module and converting the amplified temperature analog signal into a temperature digital signal by the analog-to-digital conversion module;
(15) by means of the control logic module, determining whether the temperature digital signal reaches a set temperature threshold value, if yes, go to next step, if no, return to step (2);
and (16) inactivating the chip and providing high temperature or low temperature protection.
